(19) United States
(12) Patent Application Publication
(10) Pub. No.: US 2004/0142414 A1
(43) Pub. Date: Jul. 22, 2004

(54) Production of heterologous protein in a minimal culture medium

(75) Inventors: Guillermo Gimenez Gallego, Madrid (ES); Carlos Fernandez Tornero, Grenoble Cedex (FR); Alvaro Ramon Gonzalez, Madrid (ES); Javier Varela Espinosa, Madrid (ES); Jose Luis Alonso Lebrero, Madrid (ES); Juan Pablo Pivel Ranieri, Pozuelo de Alarcon (ES)

Correspondence Address: Richard P. Berg, Esq., c/o LADAS & PARRY, Suite 2100, 5670 Wilshire Boulevard, Los Angeles, CA, 90036-5679, US

(73) Assignee: INDUSTRIAL FARMACEUTICA CANTABRIA, S.A.

(21) Appl. No.: 10/346,978

(22) Filed: Jan. 17, 2003

(51) Int. Cl. C12N 1/21; C12N 15/74

(52) U.S. Cl. 435/69.1; 435/320.1; 435/252.3

(57) ABSTRACT

The process to produce heterologous proteins in a minimal culture medium consists of (i) transforming a bacterial strain that is secretary in a minimal culture medium with a plasmid that contains the coding sequence of said heterologous protein, (ii) culturing said strain in a minimal culture medium, and (iii) recovering the heterologous protein. The bacterial strain that is secretary in a minimal culture medium can be obtained by means of a process that comprises (a) transforming bacteria adapted to grow in a minimal culture medium with a plasmid that comprises a DNA sequence that encodes for a protein that is toxic for a bacteria when it accumulates in the cytoplasm and whose synthesis is coupled to the secretion into the periplasm, (b) culturing said transformed bacteria in a minimal culture medium, and (c) selecting the surviving bacteria. It is applicable in the production of proteins of interest in bacteria that are secretary in a minimal culture medium.

### FIELD OF THE INVENTION

The invention is related, in general, to the expression of heterologous proteins in a minimal culture medium. Specifically, the invention relates to a plasmid comprising a DNA sequence that encodes for a toxic protein for the bacteria when it is accumulated in the cytoplasm and whose synthesis is coupled to the secretion into the periplasm and its applications, e.g. in the selection of secretary bacterial strains in a minimal culture medium that may be transformed to express products of interest in an minimal culture medium.

### BACKGROUND OF THE INVENTION

The expression of heterologous proteins in E. Coli is a method to obtain proteins of very diverse origin. Its advantage against other methods is its easy handling of the E. Coli cultures and the relatively large quantities that can be obtained. (Hockney R C. Recent developments in heterologous protein production in Escherichia coli. Trends Biotechnol. 1994 November; 12(11): 456-63; Grisshammer R., Tate C G. Overexpression of integral membrane proteins for structural studies. Q Rev Biophys. 1995 August; 28(3): 315-422).

In the E. coli cytoplasm, the cysteine residues are always maintained reduced by means of two systems, that of thioredoxin and that of glutation/glutaredoxin. This has the consequence that dispulphur bridges are rarely formed. When numerous proteins are expressed, especially if they are proteins from eukaryotic organisms, this peculiarity of E. coli is a serious drawback which is translated in that the proteins are synthesized denatured. Although processes of renaturalization have been disclosed, these processes are extremely expensive, very inefficient and, in general, not very applicable to industrial processes for the type of waste products produced.

An alternative is to couple the peptide synthesis to its excretion into the E. coli periplasm where it is disclosed that there is catalyst system for the formation of disulphur bridges and isomerization. In this context, the proteins easily find their correct three-dimensional structure. This method has the drawback, with a view to the production of high quality protein, that it only works correctly in rich mediums (with yeast extract), in accordance with that disclosed in the bibliography. The protein synthesized in these conditions easily comes into contact with very heterologous chemical compounds that can modify it in various forms. Furthermore, these compounds are at times very difficult to eliminate from the final preparation, which can compromise the pharmacological use of the proteins prepared by these processes and the high-resolution biochemical and physio-chemical studies.

Normally, the expression of proteins with plasmids with secretion sequences is not possible in minimal culture media with the current strains, e.g. E. coli BL21 transformed with Pin II[-omp) plasmids, the use of rich media (of the LB type) being necessary.

### SUMMARY OF THE INVENTION

In general, the invention deals with the problem of providing a system for the expression of heterologous proteins in a minimal culture medium.

The solution provided by this invention is based on the creation of a bacterial strain that secretes heterologous protein in a minimal culture medium by means of the transformation of a bacteria adapted to grow in a minimal culture medium with an expression plasmid which consists of a DNA sequence that encodes for a protein that is toxic for the bacteria when said protein accumulates in the cytoplasm and whose synthesis is coupled to the secretion into the periplasm. The plasmid comprises a repressor system of the toxic protein synthesis which prevents the production of the protein in normal culture conditions. This repressor system can be inhibited and the protein synthesis activated by using an activator of the promoter or promoters which target the start of the cDNA transcription that encodes for the protein of interest. Likewise, the plasmid comprises a gene that gives the bacteria resistance to an antibiotic, e.g. ampicillin, and permits said bacteria to grow in culture media in the presence of said antibiotic. As a consequence of this double treatment (induction of protein synthesis and bacterial growth in selective conditions), the bacteria which do not carry the expression plasmid and those which do not possess an efficient system to expulse the protein from the cytoplasm are eliminated, thus being able to select the bacterial strains which efficiently expulse the toxic protein and which grow in a medium with minimum needs. Subsequently, the plasmid that encodes for the toxic protein is eliminated thus obtaining a stable bacterial strain that can once more be transformed with an expression plasmid coupled to the expulsion of the protein from the cytoplasm and which encodes for a protein of interest (either toxic or non-toxic for the bacteria).

The creation of a strain of E. coli capable of growing in a minimal culture medium while simultaneously being capable of producing and secreting the protein into the culture medium permits purifying the protein of interest from a much cleaner original extract, marking the protein with ¹³C and ¹⁵N to be analyzed by NMR techniques with heavy atoms with a view to the use of anomalous diffraction in studies with x-rays or with radioactive isotopes for pharmodynamic studies. Furthermore, obtaining the protein in a minimal culture medium provides many advantages for its pharmacological and biochemical use, and with an appreciable production yield which facilitates its use and makes the process profitable.

### BRIEF DESCRIPTION OF THE DRAWINGS

FIG. 1 is a schematic representation of the pRat-ompA (pompA)I (pompA-1), pompA-2 and pompA-3 vectors.

FIG. 2 schematically represents the construction of the pompA-1 plasmid.

FIG. 3 schematically represents the construction of the pompA-2 plasmid.

FIG. 4 schematically represents the construction of the pompA-3 plasmid.

FIG. 5 illustrates the high production and secretion into the AM55 protein cultures medium, determined by means of electrophoresis in the presence of sodium dodecyl sulphate in polyacrylamide gels (SDs-PAGE), in E. coli cells derived from the super-secretary (BL21ss) strain (BL21(DE3) in comparison with the production of said protein in E. coli cells derived from the non-super-secretary (BL21) strain (BL21(DE3).

FIG. 6 illustrates the expression of the pompA-AM55 in BL21 (path 1) E. coli cells, determined by means of SDS-PAGE, compared with the expression of pompA-AM55 in cured BL21 E. coli cells that are retransformed with pompA-Am55 (path 2).

FIG. 7 illustrates the expression of pompA-AM55 and the production of the AM55 protein, determined by means of SDS-PAGE, in E. coli cells derived from the super-secretary (BL21(DE3) stock cultured in M9×3+ casaminoacids medium and in LB(LB) medium.

FIG. 8 illustrates the production of the C-Lyt protein, determined by means of SDS-PAGE, in E. coli cells derived from the BL21(DE3) strain cultured in M9×3+ casaminoacids at different times [FIG. 8A]; the expression of pompA-C-Lyt in E. coli cells derived from the BL21(DE3ss) strain cultured in M9(M9) medium and M9×3+casaminoacids (M9×3+aa) [FIG. 8B]; and the expression of pompA-C-Lyt in E. coli cells derived from the BL21(DE3)ss strain cultured in LB(LB) medium and in M9×3+casaminoacids (M9×3+aa) for 24 hours [FIG. 8C].

FIG. 9 illustrates the expression of the pompA-C-Igf-bp4 (pompA-c-bp4) and the production of the protein c-lgf-bp4 (c-bp4), determined by SDS-PAGE in E. Coli cells derived from the BL21(DE3)ss stock cultured in LB (LB) medium, in M9×3+casaminoacids (M9×3+aa) medium and in M9 (M9) medium, for 24 hours, at various IPTG concentrations.

FIG. 10 is an immunoblot of an expression sample of the pompAFgf-b with a specific antibody [FIG. 10B].

### DETAILED DESCRIPTION OF THE INVENTION

In a first aspect, the invention relates to a plasmid, hereinafter plasmid of the invention, which consists of:

(i) a DNA sequence that encodes for a protein which is toxic for a bacteria when said protein accumulates in the cytoplasm of said bacteria, and

(ii) a DNA sequence that contains the secretion sequence of a protein.

The protein that is toxic for a bacteria when it accumulates in the cytoplasm can be any protein that, when it accumulates, is toxic for the bacteria, i.e. causes the death or inhibition of bacterial growth, e.g. the bovine ADP/ATP translocase, the Bacillus PS 3 (membrane)/H+ alanine transporter, the bovine phosphate transporter, etc.

The DNA sequence that contains the secretion sequence of a protein can be any DNA sequence capable of targetting the secretion of a protein outside the cell, e.g. a DNA sequence that contains the secretion sequence of the outer membrane protein (ompA: outer membrane protein A) or periplasmic protein (PhoA: periplasmic phosphatase alkaline). The use of a DNA sequence that contains the secretion sequence of a protein permits the secretion of the protein to the extracellular medium. The presence of disulphur bridge oxydases and isomerases in the periplasm, together with the extracellular medium, due to its different red-ox potential with respect to the intracellular (much more oxydizing than the latter), facilitates the formation of the disulphur bridges and, therefore, the correct folding of the secreted proteins, which greatly helps to obtain proteins in their soluble form.

The plasmid of the invention further consists of a gene sequence that gives resistance to antibiotics, e.g. a gene which makes the bacteria resistant to an antibiotic e.g. ampicillin, so that it permits said bacteria to grow in culture media in the presence of said antibiotic and to select the transfected strains.

The plasmid of the invention may further contain a system that represses/activates the synthesis of said protein that is toxic for a bacteria when it accumulates in the cytoplasm of said bacteria which prevents the production of the protein in normal culture conditions. This repressor system can be inhibited and the synthesis of the protein activated when an activator of the promoter or promoters that target the start of the cDNA transcription that encodes for the protein of interest is used. In a specific embodiment, said repressor/activator system for the synthesis of said protein that is toxic for a bacteria when it accumulates in the cytoplasm of said bacteria comprises a Lac promoter, e.g. a T7 Lac RNA polymerase system which strictly regulates the synthesis of said protein that is toxic for a bacteria when it accumulates in the cytoplasm of said bacteria, or a system based on the changes of temperature or metabolic state of said bacteria, such as a repressor/activator system for the synthesis of said toxic protein which comprises a promoter modulated by arabinose or tryptophan.

The plasmid of the invention may further contain a DNA sequence containing a recognition site for a protease such as 3C protease.

In a specific embodiment, the plasmid of the invention may also contain, if so desired, an enzymatic cut site, e.g. the Nael site, immediately after the site where the peptidase cuts that permits secreting the protein into the culture medium.

The plasmid of the invention can be obtained by means of conventional genetic manipulation techniques known by those skilled in the art. Examples 1-3 attached to this description disclose the obtaining of plasmids identified as pRat-ompA-1 (pompA-1), pRat-ompA-2 (pompA-2), pRat-ompA-3 (pompA-3).

The pompA plasmid is based on the P-RAT4 plasmid [Peränen J., Rikkonen, M., Hyvönen, M., and K\ddot{aa}riänen, L.T7 Vectors with a modified T7 lac promoter for expression of proteins in Escherichia coli. Analytical Biochemistry (1996) 236:371-373], pUC-type plasmid, with a promoter-repressor system called T7lac system [Dubendorff H., J. Mol Biol. (1991) 219:45-59 and 61-68 (Sn2)]. This system consists of the lac 1^{q} gene and the promoter called T7lac which controls the expression of the heterologous protein. The T7lac and lacUV5 promoters are repressed by the lac repressor protein (encoded by the lac 1^{q} gene). The lacUV5 is recognised by the E. coli RNA polymerase, unlike the T7lac that is only recognised by the T7 RNA polymerase. The lac repressor is active until the time when the (IPTG) lac inductor is added. Thereafter, the T7 RNA polymerase begins to be expressed which in turn recognises the T7lac promoter (also unrepressed due to the inactivity of the lac repressor) and transcribes the heterologous protein gene. This double control of the transcription of the heterologous gene permits using the system for all types of proteins (even those which may be toxic for the bacteria) as there is zero expression of the heterologous protein until the IPTG is added. The plasmid also possesses the encoding gene for the β-lactamase which gives the bacteria that possess it resistance to ampicillin. This permits the selection of positive transformer strains (in the construction period of the recombinant strain) and by adding it to the expression medium possible contaminations or the appearance of strains of the same E. coli that have lost the plasmid are avoided.

The creation of the plasmid of the invention arises from the necessity to express proteins, e.g. eukaryotic proteins, in a heterologous form, for their subsequent purification, crystallization, and determination of the three-dimensional structure by techniques of X-ray diffractometry or NMR and the relevant tests for the complete characterization of their structure and biological function. The crystallization of a protein largely depends on its purity. For this reason, the use of a secretion expression vector is interesting as the culture medium contains much fewer proteins than the inner cells. The obtaining of expression strains capable of producing the heterologous protein in appreciable amounts in minimal culture media is also interesting as, with this, the protein can be marked with ¹⁵N or ¹³C (necessary to resolve the structure by NMR) and their purification starting from a much cleaner original medium (both in other proteins and other non-proteic contaminants, such as pigments, which appear in rich media and are difficult to eliminate in the purification).

To characterize the utility of the plasmid of the invention, said plasmid has been used to attempt the expression of proteins of very varied types, both in origin and characteristics.: Proteins of human origin (basic Fgf, C-Igf-bp4, N-Igf-bp4, Fgf receptor II (IgfIIIc), animal (Homothorax), plant (Am55) and bacterial (C-Lyt), some being of small size (basic Fgf, C-Lyt, N and C-Igf-bp4 and AM55, all less than 25 Kda) and others of greater size (Homothorax and Fgf receptor, of approximately 50 Kda). Some of them have all their Cys free (basic Fgf) and others have them forming disulphur bridges or not (C-Lyt, C and N Igf-bp4, Fgf-receptor and AM55) [see Example 5].

In another aspect, the invention relates to a bacteria that comprises the plasmid of the invention; preferably, said bacteria is capable of growing in a minimal culture medium. In a specific embodiment, said bacteria is of an Escherichia coli strain adapted to grow in a minimal culture medium.

To create the secretary bacterial strain in a minimal culture medium provided by this invention, a method has been developed that consists of transforming a bacteria with a plasmid of the invention, specifically with an expression vector that encodes for a protein that is toxic for the bacteria when it accumulates in the cytoplasm and whose synthesis is coupled to the secretion into the periplasm. The bacteria is adapted to grow in a minimal culture medium comprised, at least, of one carbonated source and another nitrogen one and can be selected, in conditions in which the synthesis of the protein encoded in the plasmid is induced, by means of the use of antibiotics in a culture medium when it is transformed by a plasmid which permits resistance to said antibiotics or by the use of other forms of selection (temperature, etc.).

As a consequence of this double treatment (induction of protein synthesis and bacterial growth in selective conditions), the bacteria which do not carry the expression plasmid and those that do not possess an efficient system to expulse the protein from the cytoplasm, are eliminated. In this way, bacterial strains which efficiently expulse the protein and which grow in a medium with minimum requirements are selected. The toxic protein-encoding plasmid is subsequently eliminated and after the elimination of said plasmid, a stable strain is obtained that can be newly transformed with an expression plasmid coupled to the expulsion of the protein from the cytoplasm and which encodes a protein of interest, whether toxic or non-toxic, for the bacteria.

Therefore, in another aspect, the invention relates to a process to obtain a secretary bacterial strain in a minimal culture medium, which comprises:

a) transforming bacteria adapted to grow in a minimal culture medium with a plasmid of the invention,

b) Culturing said transformed bacteria in a minimal culture medium with an antibiotic under conditions which permit the expression of said protein that is toxic for the bacteria when it accumulates in the cytoplasm of said bacteria, and

c) Selecting the surviving bacteria.

In principle, any bacteria adapted to grow in a minimal culture medium can be suitable to put the process provided by this invention into practice. In a specific embodiment, said bacteria belong to an E. coli strain adapted to grow in a minimal culture medium. The culture conditions will depend, amongst other factors, on the bacteria chosen for the embodiment of the process. The bacteria which express the protein that is toxic for the bacteria when it accumulates in the cytoplasm of said bacteria, and cannot excrete it to the outside, are incapable of surviving. The selection of the surviving bacteria can be made by any conventional technique known by those skilled in the art, e.g. by means of selection by antibiotics.

If so desired, the process to obtain the secretary bacterial strain in a minimal culture medium further consists of the stage of eliminating the plasmid that encodes for said toxic protein of the selected surviving bacteria. The elimination of said plasmid can be carried out by any conventional technique by those skilled in the art, e.g. the methods disclosed in laboratory manuals amongst which are found (Grishammer R., Tate C G. Overexpression of integral membrane proteins for structural studies. Q. Rev. Biophys. 1995. August:28(3):315-422; Di Donato A., De Nigris M., Russo N., Di Biase S., D'alessio G. A method for synthesizing genes and cDNA's by the polymerase chain reaction. Anal. Biochem. 1993 July:212(1):291-3; Wolfson J S., Hooper D C, Swartz M N, Swartz M D, McHugh G L. Novobiocin-induced elimination of F'lac and mini-F plasmids from Escherichia coli. J. Bacteriol. 1983 December:156(3):1165-70; Uhlin B E. Nordstrom K. Preferential inhibition of plasmid replication in vivo by altered DNA gyrase activity in Escherichia coli. J. Bacteriol. 1985 May:162(2):855-7; Lockshon D. Morris D R Positively supercoiled plasmid DNA is produced by treatment of Escherichia coli. With DNA gyrase inhibitors. Nucleic Acids Res. 1983 May 25:11(10):2999-3017). After the elimination of this plasmid, a stable strain which can be newly transformed with an expression plasmid coupled to the expulsion of the protein from the cytoplasm and which encodes a protein of interest, whether toxic or non-toxic for the bacteria, is obtained.

In another aspect, the invention relates to a bacterial strain selected from the group formed by the strains which have the characteristics of the cultures deposited in the CECT with access numbers CECT 5700, CECT 5701, CECT 5072 or CECT 5073, or a variant thereof which has the capacity to grow in a minimal culture medium and secrete a protein encoded by a DNA integrated in a plasmid into said medium. In a specific embodiment, said bacterial strain is derived from E. coli. The bacterial strains deposited in the CECT are identified in the section relating to the Deposit of Biological Matter.

In another aspect, the invention relates to a process to obtain a product of interest which consists of:

a) transforming a secretary bacterial strain in a minimal culture medium which can be obtained by means of the process provided by this invention, or a bacterial strain provided by this invention, with a plasmid which contains a DNA sequence that encodes for a product of interest; and

b) culturing said secretary bacterial strain in a minimal culture medium, under conditions which permit the expression of said product of interest and its secretion into the culture medium.

In principle, any bacterial strain capable of growing in a minimal culture medium, regardless of the process to obtain it, may be suitable to put this process for the production of products of interest into practice. In a specific embodiment, said bacterial strain is an E. coli strain, secretary in a minimal culture medium.

The culture conditions will depend, among other factors, on the bacterial strains chosen for the embodiment of the process. The product of interest, expressed and secreted into the minimal culture medium, if so desired, can be recovered from it by any conventional method known by those skilled in the art. In that case, said process further comprises the stage of recovering the product of interest from the culture medium [stage c)]. Alternatively, the supernatant of the bacterial culture, which contains the product of interest, can be industrially used as it is, or instead, a fraction of it, without the need to isolate the product of interest.

In principle, any product of interest can be obtained by means of the process provided by this invention. By way of illustration, said product of interest can be a cytokine, e.g. tumoral necrosis factor (TNF), and interleukine (IL), such as IL-10, etc; a chemokine e.g MIP-1-beta (MIP-1:macrophage inflammatory protein), MCP-1 (MCP-1: macrophage chemoattractant protein 1), etc; a protein to fuse with human antibody Fc structures, e.g. TNFR-Fc, IL-IR antag-Fc, etc; a peptide with anti-gene characteristics which permits its use for purposes of vaccinations or diagnostics by means of, e.g. the use of ELISA techniques, etc.

Finally, in another aspect, the invention relates to the use of a protein that is toxic for a bacteria when it accumulates in the cytoplasm of said bacteria in the selection of secretary bacteria.

The following examples illustrate the invention and should not be considered as limiting its scope.

### EXAMPLES

### Materials and Methods

1. Bacterial Strains and Plasmids.

In the development of this invention, the bacterial strains and the plasmids listed in Tables IA and IB have been used.

TABLE IA
Bacterial strains

| | Relevant genotype | |
|---|---|---|
| E. coli strains | or phenotype | Reference |
| TG1 | F′ (lacIqlacz M15) | Gibson, 1984 |
| BL21(DE3) | B F-dem omp T hsdS | Phillips et al., |
| | (r_{B}-m_{B})gal Λ(DE3) | 1984; Borck et |
| | | al., 1976 |
| BL21(DE3)ss | B F-dem omp T hsdS | |
| | (r_{B}-m_{B})gal Λ | |
| | (DE3)ss | |

Phillips et al., 1984: Phillips T A. VanBogelen R A. Neidhardt F C. Ion gene product of Escherichia coli is a heat-shock protein. J. Bacteriol. 1984-Jul; 159(1): 283-7)
Borck et al., 1986: Borck K. Beggs J D. Brammar W J. Hopkins A S. Murray N E. The construction in vitro of transducing derivatives of phage lambda- Mol. Gen. Genet. 1976 Jul 23: 146(2): 199-207)

TABLE 1B
Plasmids

| | Relevant genotype | |
|---|---|---|
| E. coli strains | or phenotype | Reference |
| PUC19 | Ap R, lac Z | Yanisch-Perron et |
| | | al., 1985 |
| PUC18 | Ap R, lac Z | Yanisch-Perron et |
| | | al., 1985 |
| Prat4 | AP R, T7 promoter | Peranen et al., |
| | | 1996 |
| Prat-ompA | Ap R T7 promoter, | Yanisch-Perron et |
| | ompA sequence, stop | al., 1985 |
| | sequence, His6 | |

Yanisch-Perron et al., 1985; Yanisch-Perron c. Vieja J., Messing J: Improved Ml3 phage cloning vectors and host strains: nucleotide sequences of the Ml3mp 18 and pUC19 vectors. Gene. 1985: 33(1): 103-19.
Peränen et al., 1996: Peränen J., Rikkonen, M., and Kääriänen, L. T7 vectors with a modified T7lac promoter for expression of proteins in Escherichia coli. Analytical Biochemistry (1996) 236: 371-373.

2. Preparation of the Plasmids.

To obtain the plasmids from the E. coli strains, a method that follows the protocol attached to the Kit for the extraction of DNA from plasmids was used. High Pure Plasmid Isolation Kit (Boehringer Mannheim).

3. Manipulation of DNA with Enzymes Commonly Used in Molecular Biology.

The restriction enzymes used in the manufacture of the constructions were those listed hereunder:

Amersham Xbal and EcoRV

Biolabs NaeI, HpaI, Mscl, A1wNI and SmaI.

Boehringer Mannheim EcoR1, Hind III, Nrul and Stul.

MBI Fermentas HindIII and EcoRI The T4-DNA-ligase and T4-polynucleotide-kinase enzymes were obtained from the Boehringer Mannheim company. MBI Fermentas T4-DNA-ligase was also used.

The Depp Vent enzyme involved in the PCR processes was supplied by Biolabs.

All the enzymes and their corresponding buffers were used in accordance with the instructions from the company who supplied them.

4. PCR and Oligonucleotide Synthesis

The oligonucleotides used for the PCR of the ompA synthesis were designed taking into account the triplets of the bases most favoured for their subsequent translation, when synthesizing the proteins, in E. coli and synthesized in a Gene Assembler Plus Pharmacie type synthesizer, by the Chemical Protein Service of the Centre for Biological Research C.I.B Madrid). The synthesis was carried out according to the model of oligo superposition for the synthesis of genes disclosed by Alberto di Donato et al. [Di Donato A. et al., A method for synthesizing genes and cDNA's by the polymerase chain reaction. Anal Biochem. 1993 July, 212(1):291-3]. The amplification apparatus used for the synthesis of the different genes was a PCR Robocycler 40; of the Stratagene company.

The PCR for the amplification of the genes corresponding to the various proteins cloned was carried out in the same amplification apparatus and with oligonucleotides synthesized in the Chemical Protein Service of the CIB, All the PCR were carried out with the Depp-Vent (Biolabs) enzyme.

5. DNA Sequencing

The analysis of the sequences obtained by PCR was carried out by the DNA Sequencing Service of the Biological Research Centre (CIB) using for this the Perkin Elmer ABIPRISM 377 automatic sequencer. The sequencing was carried out in the pUC19 or pUC18 plasmids. The product of the PCR was directly cloned in the pUC plasmids, linearized with SmaI.

6. Electrophoresis in Agarose Gels.

In the electrophoresis of the different DNA and plasmid fragments, 0.8% and 2% agarose gels in TAE were used, the same buffer being used as electrolyte.

As loading buffer of the sample in the gel, a solution comprised of 15% Ficoll 400, 0.2%(p/v) bromophenol blue and 0.25% Xilencianol in a ratio of 1/10 sample volume.

The electrophoresis was carried out at a constant voltage of 90-120 volts, for 30 to 50 minutes, in cuvettes provided for that purpose. The source of electrophoresis used was BIORAD POWR PAC 300.

To view the different DNA fragments after electrophoresis, the gels were dyed with a 10 mg/ml solution of ethydium bromide (Boehringer Mannheim) and exposed under ultraviolet light.

The molecular weight markers used as a size reference were DNA Molecular Weight Marker II (Boehringer Mannheim) and markers of 100 base-pairs ladders (pb) (100 Base-pair Ladder) of Pharmacia Biotech.

7. Isolation and Purification of the DNA Fragments.

The fragments resulting from the digestion of the DNA by the different restriction enzymes were isolated and purified in agarose gels of low melting point, using the Millipore Ultrafree-DA commercial Kit.

8. Culture Media and Conditions.

For the growth of E. coli in a solid medium in all the construction processes of the expression plasmids, the LB medium with agar, 1.5% (w/v) was used.

The media used for the growth of E. coli in liquid culture and the expression of the different transformer proteins is listed in Table II. The antibiotic used to give resistance to the E. coli strains was ampicillin, in a final concentration of 200 mg/ml. This antibiotic was prepared in water in a concentration of 100 mg/ml, filtered (millex millipore 0.22 μm and aliquoted in sterility in eppendorf 1.5 ml.

The bacteria used in the inoculation of the cultures comes from cultures that have been frozen in carbonic snow, with 15% (w/v) glycerol; and kept at −80° C., in a REVCO chest freezer.

The growth of the strains was monitored by turbidimetry at 600 nm with a Guildford Microsampler 300-N type spectrophotometer.

TABLE II
Composition of the LB, M9 × 3 + casaminoacids and M9 media

| | Quantity | | Company |
|---|---|---|---|
| LB Medium | | | |
| composition | | | |
| Bacto-tryptone | 10 | g | DIFCO |
| Yeast extract | 5 | g | Pronadisa |
| Sodium chloride | 1 | g | Merck |
| Glucose | 1 | g | Merck |
| Milli-Q water | Up to 1,000 | ml | |
| M9 × 3 + casaminoacids | | | |
| Medium composition | | | |
| M9(× 19) Medium | 150 | ml | |
| Ca/Mg | 15 | ml | |
| Vitamin B1 (1 mg/ml) | 15 | ml | |
| 20% glucose | 30 | ml | |
| Casaminoacids | 50 | ml | |
| Milli-Q water | 190 | ml | |
| M9(× 10) Composition | | | |
| PO₄HNa₂ 12 H₂O | 88.25 | g | Merck |
| KH₂PO₄ | 15 | g | Merck |
| NH₄Cl | 5 | g | Merck |
| Sodium chloride | 2.5 | g | Merck |
| Milli-Q water | 500 | ml | |
| Ca/Mg composition | | | |
| Cl₂Ca.2H₂O | 147 | mg | Merck |
| So₄Mg·/H₂O | 2.46 | mg | Merck |
| Milli-Q water | Up to 100 | ml | |
| Glucose composition | | | |
| Glucose | 20 | mg | |
| Milli-Q water | Up to 100 | ml | |
| Casaminoacids | | | |
| composition (× 10) | | | |
| Casaminoacids | 20 | g | DIFCO |
| Milli-Q water | 500 | ml | |
| M9 medium | | | |
| composition | Amount | | Company |
| M9(× 10) Medium | 50 | ml | |
| Ca/Mg | 5 | ml | |
| Vitamin B1 (1 mg/ml) | 5 | ml | |
| 20% glucose | 10 | ml | |
| Milli-Q water | 430 | ml | |

9. Competence and Transformation of Bacterial Strains.

The competent cells were obtained following the method disclosed by Lederber & Cohen (Lederberg E M. Cohen S N. Transformation of Salmonella typhimurium by plasmid deoxyribonucleic acid. J. Bacteriol. 1974 September; 119(3): 1072-4) or the calcium chloride method. The bacterial strains to which the competence process was applied were TG1, BL12(DE3)ss (Phillips T A. VanBogelen R A. Neidhardt F C. Ion gene product of Escherichia coli is a heat-shock protein. J.Bacteriol. 1984-July; 159(1):238-7; Borck K. Beggs J D. Brammar W J. Hopkins A S. Murray N E. The construction in vitro of transducing derivatives of phage lambda—Mol. Gen. Genet. 1976 July 23:146(2): 199-207). The competent cells were conserved at −80° C., with 15% (v/v) glycerol. The aliquots were 300 ml, in 1.5 ml eppendorf tubes.

Before the transformation process, each aliquot from competent cells was removed from the −80° C. freezer and kept in ice for 15 minutes, to defrost.

Next, the DNA transformer was inoculated by pipette, inside the phial containing the competent cells. After 15 minutes in ice, the cells were subjected to a thermal shock for 4 minutes at 37° C., at the end of which they were again placed in ice for 5 minutes. Then, 1 ml of LB was added as expression medium and kept for 1 hour in a bath at 37° C. After this time, the cells, already transformed, were plating in LB Agar Ampicillin plate. The solutions used were 1:10 (15 ml of the transformation volume+135 ml LB); 1× (150 ml of transformation volume); and 10× (the remaining transformation volume was centrifuged for 1 minute at 15,000 rpm in an eppendorf 5415 C centrifuge and the cells were re-suspended in 150 ml LB). The cells used in all the transformations carried out during the construction of the different plasmids were TG1. The cells of the BL21(DE3) and BL21(DE3(ss) strains were used to transform the finished expression plasmids and perform the relevant recombinant protein production tests on them.

10. Curing of the BL21(De3)ss Strain.

Novobiocin is a specific inhibitor of the DNA Gyrase type B Bacteria. (Conzzarelli N R. DNA gyrase and supercoiling of DNA. Science 1980 February 29:207/(4434):953-60. Gellart M. Fisher L M. Ohmori H. O'Dea M J. Mizuuchi K. DNA gyrase: site specific interactions and transient double strand breakage of DNA. Cold Spring Harn Symp Quant Biol., 1981:45 Pt 1:391-8). In E. coli, the DNA gyrase introduces negative DNA supercoiling. The degree of supercoiling depends on a dynamic balance between both antagonistic enzymatic activities: that of the DNA gyrase and that of the DNA topoisomerase I. (DiNardo, S. Voelkel K A. Stenglanz R. Reynolds A E. Wright A. Escherichia coli DNA topoisomerase I mutants have compensatory mutations in DNA gyrase genes. Cell 1982 November:31(1): 43-51; Prüss G J. Manes S H. Drlica K. Escherichia coli topoisomerase I mutants: increased supercoiling is corrected by mutations near gyrase genes. Cell 1982 November:31(1):435-42). The culture of E. coli strains in media that contain said antibiotic in concentrations between 25 and 200 mg/ml permits their growth but induced the elimination of the plasmids that they may possess (Wolfson J S. Hooper D C. Swartz M N, Swartz M D, McHugh G L. Novobiocin-induced elimination of F'lac and mini-F plasmids from Escherichia coli. J. Bacteriol. 1983 December:156(3):1156-70; Uhlin B E. Nordstom K. Preferential inhibition of plasmid replication in vivo by altered DNA gyrase activity in Escherichia coli. J. Bacteriol. 1985 May:162(2):855-7) This is caused because the plasmids undergo positive supercoiling due to the inhibition of the DNA gyrase B, which is more specific for plasmids than for chromosomic DNA. (Lockshon D. Morris D R. Positively supercoiled plasmid DNA is produced by treatment of Escherichia coli. With DNA gyrase inhibitors. Nucleic Acids Res. 1983 May 25:11(10):2999-3017) and this interferes in the replication of the plasmids (Uhlin B E. Nordstom K. Preferential inhibition of plasmid replication in vivo by altered DNA gyrase activity in Escherichia coli. J. Bacteriol. 1985 May:162(2):855-7).

The strain is treated with 5 stages in LB dish with novobiocin in concentrations of 100 mg/ml, finally obtaining a strain of the BL21(De3)ss cell that maintains all the characteristics of the original strain, but free of plasmids. For each successive stage, all the colonies of the dish are collected re-suspending them in 1 ml of LB. Of this 1 ml, a 1/10000 solution is prepared of which 100 ml is plating. After the 5 stages, 30 colonies were analysed, all being capable of growing in LB and incapable of growing in Lb-Amp, which indicates that all were cured. To demonstrate that it maintains the secretion characteristics, a retransformation is carried out with the same plasmid that it had before curing it and it is tested for expression. It is verified that it maintains the characteristics as the quantity of expressed protein is identical. An attempt was also made to express with the cured strain without retransforming, to completely assure that it had lost the plasmid. It is demonstrated here that no protein at all is produced.

11. Obtaining Recombinant Proteins

The proteins were obtained from culture media, in the majority of the cases being concentrated with 3 kDa ultrasette (Filtron) filters to have the protein in a much smaller volume (from the 2 or 3 initial litres, it is concentrated up to a volume of between 50 and 100 ml) which undergoes different chromotographic purifications until the pure protein is obtained.

The expression of different proteins is carried out by means of induction of the culture medium in its exponential phase (optical density of 0.8) with IPTG in a concentration of between 0.1 and 1 mM, the optimum normally being that of 0.5 mM.

12. Evaluation if the Pure Protein Concentration.

The protein quantification was carried out at 280 nm, in a Spectronic 3,000 Array (Milton Roy) spectrophotometer.

13. Protein Electrophoresis in Polyacrylamide Gels.

The electrophoresis in the presence of sodium dodecyl sulphate in polyacrylamide gels (SDS-PAGE). The packaging gel was formed by 6% polyacrylamide in Tris(0.125 M HCl, pH 6.8, SDS 0.1%, ammonium persulphate 0.33 mg/ml, TEMED 1 1 μl/ml and the separation gel by 15% polyacrylamide in 0.05 M Tris/HCl, 0.192 M glycine, SDS 0.1%.

The samples were heated to 100% for 4 minutes in the presence of a sample buffer comprised of Tris/HCl 0.1 M pH 6.6 0.1 M MgCl₂, 4% SDS, 10% glycerol, 5% DDT (15.43 mg/ml) and 0.01% bromophenol blue. The samples were then loaded in the gel to be separated by electrophoresis.

The type of gel used was in a dish (100×80×1.5 mm). The electrophoresis cuvette used was Hoefer Scientific Instruments' Mighty Small II SE 250. The electrophoresis was carried out at room temperature and at a constant voltage (120 volts).

The proteins were viewed with Coomassie R-250 blue (Swank T R. Munkres K D. Molecular weight analysis of oligopeptides by electrophoresis in polyacrylamide gel with sodium dodecyl sulphate. Anal Biochem. 1971 February:39(2):462-77).

The samples loaded in volumes above 20 ml were concentrated by using the Strataclean resin (Stratagene: Strategies in molecular biology. Kirk Nielsen et. al. 1998). When this resin is used, heating is not necessary to produce the denaturalizaton of the sample, if the sample buffer is added in a similar manner to that of the rest of the samples.

14. Immunochemical Analysis of the Protein.

14.1 Protein Transfer from a Gel to a Nitrocellulose Membrane, Electroblots.

The gels on which the protein analysis electrophoresis was then balanced with a 25 mM Tris/HCl, 20% (w/v) methanol, pH 10.4 solution for 15 minutes with gentle agitation. To assemble the transfer sandwich, the nitrocellulose membrane (Milipore GSWP 304 FO), of 85×54 mm, was balanced in this same solution, together with three layers of 3 MM paper of the same size; 6 layers of 3 MM paper were balanced with a 40 mM caproic acid solution comprised of 25 mM Tris/HCl, 20% (w/v) methanol, pH 9.4; and another 6 layers with a solution comprised of 300 mM Tris/HCl , 20% (w/v) methanol, pH 10.4. The transfers were carried out at a constant intensity of 110 mA, for 60 minutes, in a SEMI FHOR TE 70 (Semi Dry Transfer Unit, of Hoefer Scientific Instruments) transfer apparatus.

Once the transfer was completed, the nitrocellulose membrane was blocked with a solution comprised of 50 ml of TBS (Tris-Buffered Saline), 1% powdered milk; for 12 hours at 4° C.) at times, 1 hour at 37° C.). The transferred gel was dyed with Coomassie Blue R-250, as described above.

14.2 Western Blot.

To determine the basic Fgf protein, the nitrocellulose membrane was first placed in contact with an anti-protein antibody. In this way, the membrane was kept in a solution of 50 ml TBS, 0.2% powdered milk, 1% goat serum (Serum Goat, SIGMA), 200 ml specific antibody for 2 hours at room temperature and gentle agitation. Next, 3 15-minute washes with 50 ml TBS, 0.1% powdered milk removed any antibody remains that had not been bound to the protein from the membrane.

The membrane was then incubated with the primary antibody. Thus, the membrane was placed in contact with 50 ml TBS, 0.1% powdered milk, 0.4% gelatine (Gelatina Eia Purity Reagent, BIORAD), 40 ml secondary antibody (goat/anti-rabbit) conjugated with alkaline phosphatase (Anti-Rabbit IgG-whole molecule-Alcaline Phosphatase Conjugate, SIGMA) for 2 hours at room temperature and gentle agitation. Likewise, 2 15-minute washes with 50 ml TBS, 0.1% powdered milk, removed, in this case, any remains of secondary anti-bodies that had not been bound to its corresponding protein-antibody complex.

The substitution of the membrane was carried out by means of the chromogenic compounds—NBT (nitro blue tetrazolium chloride, Boehringer Mannheim) and BCIP (5-bromo-4-chloro-3-indolyl phosphate, Boehringer Mannheim), in 10 ml of Alkaline Phosphatase buffer (100 mM Tris/HCl, 100 mM Nacl, 5 mM MgCl₂, ph 9.5).

To determine the proteins with the histidine tail, the nitrocellulose membrane is treated with a histidine anti-tail antibody as it has associated the capacity to produce colour on contact with a chromogenic solution. It is an antibody fused with an enzyme: peroxidase (Sigma). This was the method used to determine the n-Igf-bp4 protein by western blot.

### Example 1

### Construction of pRAT-ompA-1 (pompA-1)

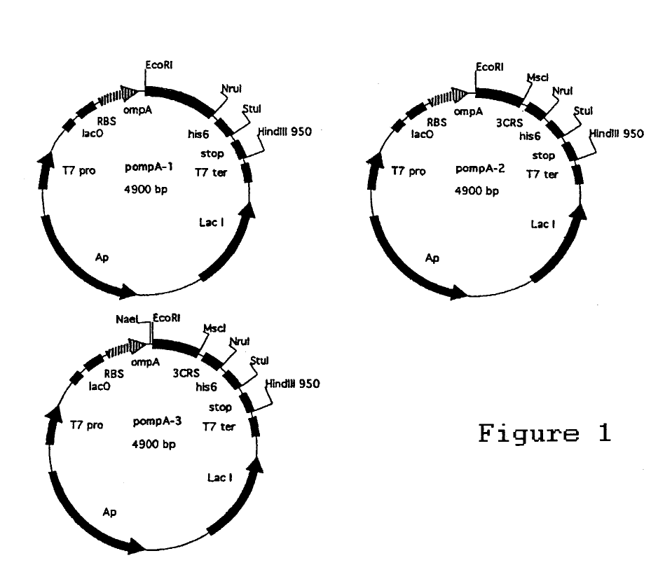

The construction of the pRAT-ompA-1 (pompA-1) plasmid [see FIG. 1] was carried out as a construction of the pompA-N-igf-bp4 plasmid, so that other proteins could be cloned from this protein. The cloning of the Fgf-b, Homothorax and AM55 proteins are illustrated.

1.1 Construction of the pRAT-ompA-N-Igf-bp4 (pompA-nbp4)

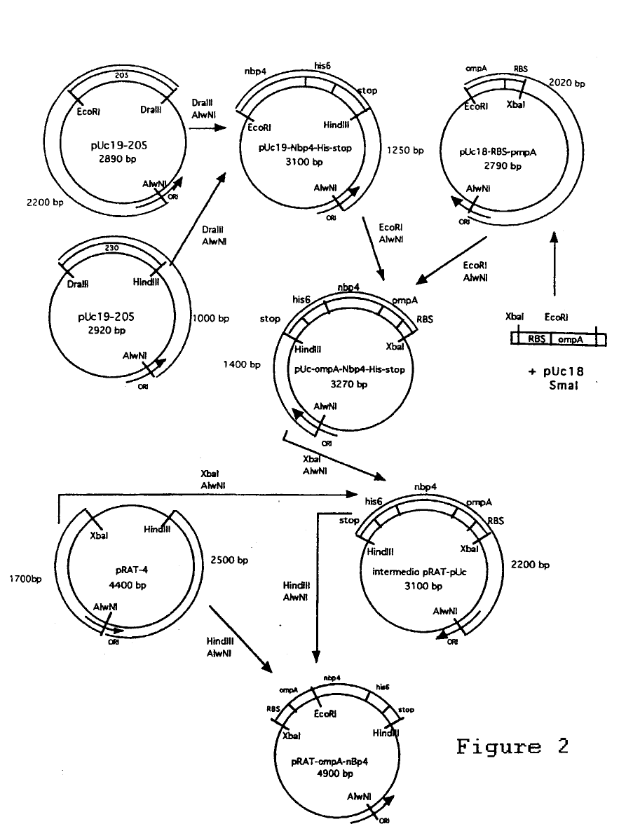

The pompA (rRat-ompA) plasmid was constructed from the pRAt-5 (Peränen J. et al., T7 Vectors with a modified T7lac promoter for expression of proteins in Escherichia coli. Analytical Biochemistry (1996) 236:371-373) and a pUc plasmid which contains all that has to be introduced to create the pompA plasmid as well as the gene that encodes the nbp-4 (pUc-ompA-nbp4-his-stop) plasmid. The complete construction process of the pompA-1 plasmid is shown in FIG. 2.

The omp-A sequence was designed by the method of overlapping oligos (Di Donato et al., A method for synthesizing genes and cDNA's by the polymerase chain reaction. Anal Biochem. 1993 July:212(1):291-3) from the secretion sequence of the ompA protein, but optimizing the codons for E. coli. It was designed for it to be cloned in the Xbal site of the p-RAT-4 plasmid for which reason it was also necessary to synthesize the binding site of the ribosome (RBS) of the vector. A distance of 14 bases was maintained between RBS and the initial codon of the translation (ATG) given that it seemed the optimum one for the expression. The product of the PCR, ompA-RBS, was cloned in pUcl8 blunt cut with SmaI with which the pUc18-RBS-ompA plasmid was obtained.

The gene that encodes the n-bp4 together with the histidine (his) and tail and stop sequences was also designed by the method of overlapping oligos optimizing the codons for E. coli but, on forming a total sequence of approximately 435 pb, it was necessary to perform it in two fragments. These fragments, of 205 and 230 pb, were directly cloned in pUc19 cut with SmaI, obtaining the pUc19-205 and pUc19-230 vectors. Through the combination of these two vectors with DraIII and AlwNI, the pUc19-nbp4-His-stop vector was obtained.

The Puc18-RBS-ompA plasmid was cut with AlwNI and EcoRI and the 1,000 pb strip was purified. The pUc18-nbp4-His-stop plasmid was cut with the same enzymes and the 2,200 pb strip was recovered. These two strips were bound obtaining the pUc-ompA-Nbp4-His-stop plasmid.

Thanks to the compatibility of the pUc and pRAT vectors, it is possible to obtain the p-RAT-ompA-Nbp4 vector by means of the construction of an “intermediate plasmid” between the two. For this, the pUc-ompA-Nbp4-His-stop plasmid was cut with Xbal and AlwNI and the 1,370 pb strip was recovered. The pRAT plasmid was cut with the same enzymes and the 2,500 pb band was recovered. This “intermediate plasmid” was cut with HindI and AlwNI as with the p-RAT-4 plasmid and 3,300 and 2,000 pb bands were recovered respectively. The resulting plasmid, purified from the positive transformer colonies, is the plasmid called pompA-nbp4.

This intermediate construction method makes it easier to handle the fragments (as strips of the gels are cut and purified by precipitation) as they are of an appreciable size, and additionally, the possibility of relegates is avoided as one works with cut, purified strips from agarose gels that are not viable as plasmids if they do not bind with another fragment, also large, that is added.

This vector (pompA-nbp4) permits cloning proteins between EcoRI and two sites that are blunt cut STU I or Nru I (depending on if the histidine tail is required or not) or between EcoRI and HindIII if the protein gene that is cloned already carries the stop sequence, with which the stop sequence of the pompA plasmid would be lost (see FIG. 2).

1.2 Construction of the pompA-Fgf-b Plasmid

Starting from a plasmid that contained the sequence that encoded the basic Fgf, a PCR was carried out so that the ends necessary to clone it in the pompA plasmid were introduced in the sequence generated. A An EcoRI site was introduced in the N-terminal end and a StuI end on the C-terminal end as, in this case, it was designed so that the product would be expressed without a histidine tail as it not necessary for the identification (due to the fact that a specific antibody exists for said protein) nor for the purification (as it is purified by means of the use of a heparin-sepharose column due to this protein's specific affinity for heparin). The product of the PCR was targeted with said enzymes, as with the vector, and the resulting fragments were bound and transformed in TGI. The construction was sequenced in the pompA plasmid.

1.3 Construction of the pompA-Homothorax Plasmid

Starting from a plasmid containing the sequence that encodes for the Homothorax protein, a PCR was carried out so that the ends necessary to clone in the pompA plasmid with the histidine tails was introduced in the sequence generated. A EcoRI site was introduced in the N-terminal end and a NruI site in the C-terminal end. The product of the PCR was originally cloned in a pUc18 plasmid where it is sequenced. From this, both this construction and the pompA plasmid were cut with the EcoRI and NruI enzymes.

1.4 Construction of pompA-AM55

The fragment obtained from the pUc18-AM55 plasmid targeted with EcoRI and with StuI is cloned in pompA targeted with the same enzymes. Therefore, the proteins lacks histidine tails as, in this case, they are not necessary for its purification or identification.

### Example 2

### Construction of pRAT-ompA-3CRS (pompA-2)

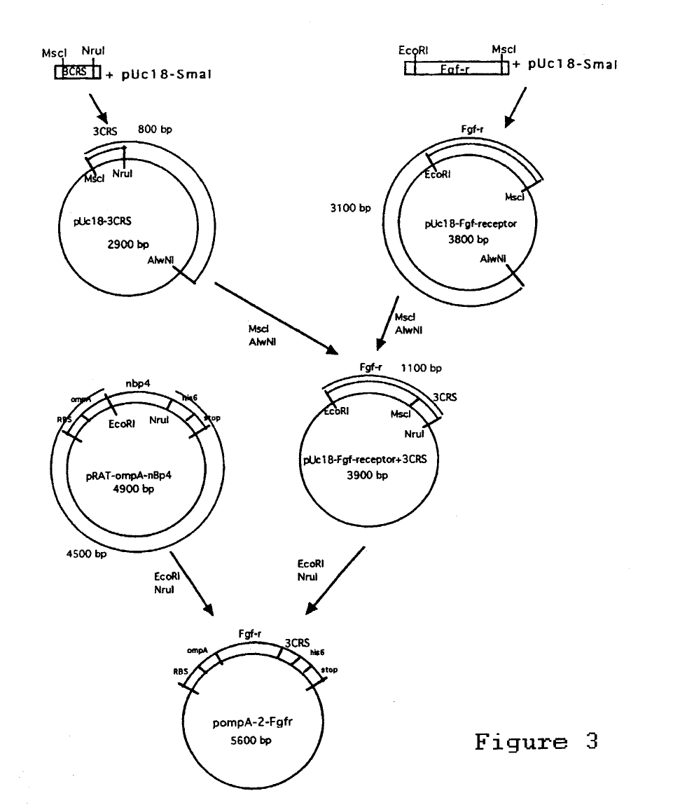

The crystallization of a protein is a process that is very sensitive to the presence of destructured amino acids. It is possible that a protein does not crystallize simply due to having a tail consisting of several amino acids which do not have an ordered secondary and/or tertiary structure. Therefore, the presence of a histidine tail could prevent these crystals from being obtained and, in consequence, would prevent the determination of the structure of the expressed protein due to X-ray diffraction, which is the basic objective for which it is obtained. To avoid this problem, while maintaining the histidine tail to purify the protein, the plasmid is redesigned so that a 3C (3CRS) protease recognition site appears before the histidine tail. In this way, the protein can be obtained with the histidine tail, be purified thanks to it, and, subsequently, the pure protein can be treated with the 3C protease so that the histidine tail is eliminated. To separate the pure protein without poli-histidine peptide tails, one only has to again pass the solution through a Ni₂ column where the peptide will be retained. The pompA-2 plasmid [see FIG. 1] was constructed with a pompA-Fgf-receptor plasmid. The construction process is shown in FIG. 3.

2.1 Construction of the pompA-Fgf-Receptor II (IgIII-c) Plasmid.

As in the previous cases (Examples 1.2, 1.3 and 1.4) a PCR was carried out from an ADNc of a Fgf receptor II protein in order to add the necessary ends, to clone in a plasmid provided by this invention, to it. Furthermore, another PCR was carried out by means of the system of overlapping oligos to redesign the plasmid so that sequence of amino acids (3CRS) remained between the histidine tail and the protein to be expressed. It was designed so that another blunt site, MscI, appeared before the 3CRS, so that once the protein to be expressed was cloned, the 3CRS site, the histidine tail, or both, can be eliminated simply by cutting and rebinding. Therefore, the cloning sites were the same as for the pompA-1 plasmid, also having the MscI site to clone the protein just before the 3CRS site (see FIG. 3).

### Example 3

### Construction of pRAT-ompA-3 (pompA-3)

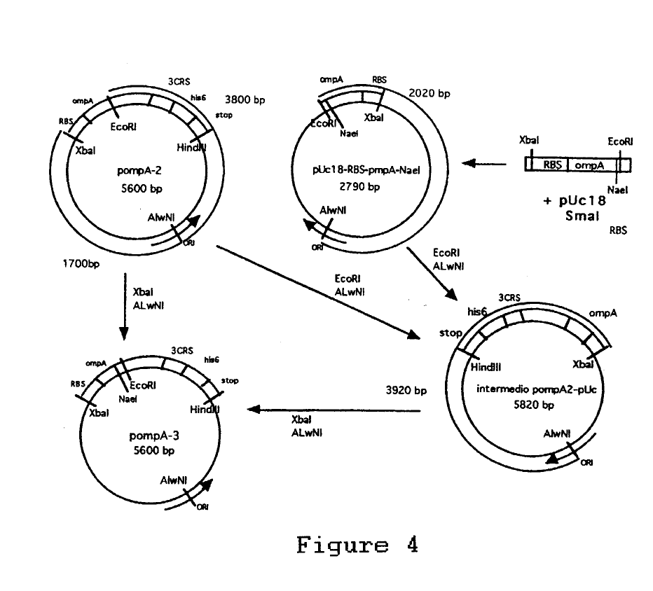

This pompA-3 plasmid [see FIG. 1] was designed so that the initial cloning site, which previously had to be EcoRI can continue to be EcoRI or a NaeI site. For this, a new PCR was carried out using the method of overlapping oligos (similar to that carried out for the construction of the pompA-1 plasmid). The product of the PCR was blunt-cloned in pCu18 and, from this, the construction was carried out in an identical manner to that of the pompA-1 plasmid construction (see FIG. 4).

The NaeI site permits cloning immediately after the site where the peptidase which cuts the ompA marking sequence is cut when the protein is expressed into the culture medium. Thanks to this, the expressed protein has the amino acid sequence that is precisely required. If the cloning is performed with EcoRI, 2 extra amino acids must be added. Furthermore, the fact that it is a blunt site permits cloning sequences cut with any enzyme that blunt cuts and it is even possible to clone a PCR fragment directly in the expression vector without it being necessary to perform intermediate steps through cloning vectors (pUc-type) or the need to cut the PCr product with restriction enzymes.

3.1 Construction of pompA-C-Igf-pb4 (pompA-Cbp4)

Starting with a construction of this same protein in another plasmid, it was cut with the HpaI and HindIII sites and bound with the vector cut with NaeI and HindIII. In this case, the construction already carried the stop sites (identical to those of the pompA plasmid) in the sequence to be inserted, but it did not carry the histidine tail as it was not necessary for its purification.

3.2 Construction of pompA-C-Lyt

Starting with the pG1-100 plasmid (García J L. García E. López R. Over production and rapid purification of the amidase of Streptoccus pneumoniae. Arch Microbiol. 1987:149(1):52-6) a PCR was carried out to clone the protein C-terminal domain. The PCR was performed so that it was possible to clone in the pompA plasmid cut with NaeI and HindIII. In this case, the stops already carried it from the old sequence. The PCR product was directly cloned in pUc19 cut with SmaI and was sequenced. From the construction, the construction was cut with HpaI and HindIII and the fragment obtained was introduced in the pompA (see FIG. 4).

### Example 4

### Selection of the BL21(De3)ss Super-Secretary Strain

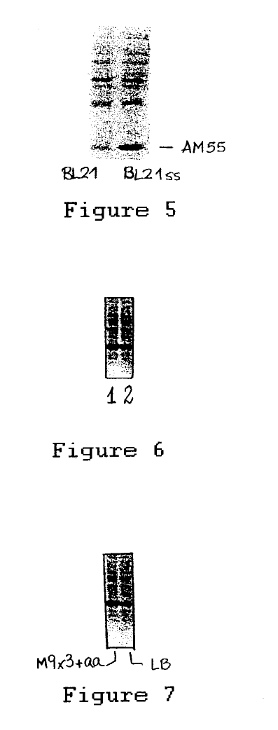

This strain derives from the BL21(DE3) strain (Phillips T A. VanBogelen R A. Neidhardt F C. Ion gene product of Escherichia coli is a heat-shock protein. J. Bacteriol. 1984-July; 159(l):283-7; Borck K. Beggs J D. Brammar W J. Hopkins A S. Murray N E. The construction in vitro of transducing derivatives of phage lambda. Mol. Gen. Genet. 1976 July 23:146(2): 199-207). When one attempts to express the AM55 protein, it is verified that it did not have a very high production. To improve the production, a BL21(DE3) strain transformed with the pompA-AM55 plasmid (Example 1.4) underwent a selection in a minimal medium culture dish with ampicillin (M9+Ap) with the (IPTG) inducer from a culture in M9 of said strain (system based on the selection of the C4(DE3) strain. The colonies appeared very slowly and were few and very small (in comparison with the control carried out starting with the same initial culture but placed in a dish in M9+Ap but without IPTG). If we analyse the expression levels of these new strains selected, two types are observed: (i) some which did not produce the protein at all, and (ii) others which produced it in an appreciably larger quantity and secreted it directly into the culture medium. The latter were collected using normal microbiological methods and were called BL21(DE3)ss (super-secretors) strains (see FIG. 5).

To be able to use said bacterial strains to express other proteins with a vector provided by this invention, it is necessary to “cure” said strains. This process consists of selecting a strain that preserves the super-secretary qualities, and, in which the pompA-Am55 plasmid has been eliminated. For this, the strain to be cured is treated with sub-lethal doses of the novobiocin antibiotic in successive stages in dishes and in a liquid LB medium (unlike the system used for the C41(DE3) which simply performed the curing by successive stages of the bacteria through culture medium free of ampicillin).

To ascertain that no plasmids are conserved by the selected strains, the same colony was placed in dishes with and without ampicillin (Ap). The cured strains grow in the dishes which do not have ampicillin and do not grow in those which do contain the antibiotic, which indicates that the cured strains do not have a ampicillin-resistant plasmid, i.e. they have totally lost the pompA-Am55 plasmid.

Furthermore, to ascertain that the super-secretary (capable of growing and producing large quantities of the required protein in a minimal culture medium and secreting it into the culture medium in a soluble form which is correctly folded) phenotype has been conserved, competent cells were produced from one of the cured colonies and the pompA-AM55 plasmid was retransformed in it. Next, an expression test was carried out in M9+Ap+IPTG in parallel with the AM55 super-secretary strain before curing it and with several of the strains obtained in the retransformation with the pompA-AM55 plasmid. The results obtained demonstrated that the protein expression was maintained (see FIG. 6).

### Example 5

### Expression of Proteins with the pompA Vector and BL21(De3)ss Cells

### Production of Protein in Relation to Times, Culture Media and IPTG Concentration

Expression tests of the different pompA-protein plasmids constructed (Examples 1-3) transforming BL21(DE3)ss cells were performed. The expression kinetics were tested in different culture media: LB, M9×3+Casaminoacids (M9×3+aa), and M9, samples were taken after different times: from 16 hours to 5 days, and different IPTF concentrations (from 0.1 to 1 mM).

5.1 AM55 Production

The Am55 protein was produced in a large quantity (after being purified, up to 30 mg per litre of original culture was obtained). The protein production was similar in this case both in M9×3+aa and LB (FIG. 7). The expression time was 24 hours.

5.2 C-Lyt Production

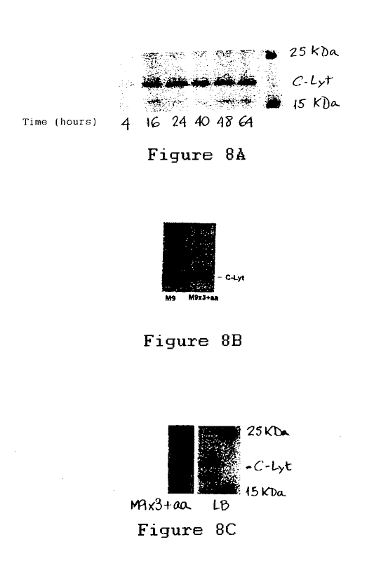

The C-Lyt protein was also produced in appreciable quantities (10 mg per litre of original culture). Maximum production was after 16 hours (FIG. 8A). The maximum production was produced in M9, whilst it is somewhat less in M9×3+aa and a good deal lower in LB, although still appreciable (FIGS. 8B and 8C).

5.3 c-Igf-bp4 (c-bp4) Production

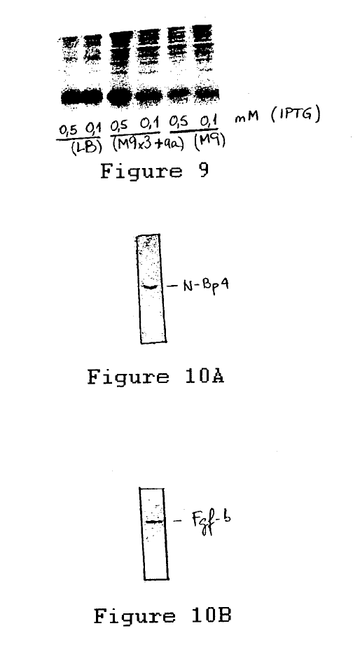

The production of the c-Igf-bp4 (c-bp4) protein was optimum after 24 hours of culture. The maximum quantity was obtained in the M9×3+aa culture (reaching the amount of 27 mg of protein per litre of original culture) and somewhat less is LB. In M9, the production was lower, although still appreciable (FIG. 9).

The best expression times in M9×3+aa seemed to be around 24 hours in the majority of cases. The amount of protein did not increase considerably with greater time, but the contaminants did (both proteic and non-proteic) due, above all, to the lysis produced in the bacterial cells by them pouring their intracellular contents into the medium. After long times, the presence of a non-proteic compound which gave a yellow colour to the medium and which was problematic due to being unspecifically retained in the chromatography columns tested and hinder to quite a degree the purification of the proteins, especially increased. The optimum IPTG concentration in the expressed protein tests was of 0.5 mM (data shown for cbp4 in FIG. 9).

On the contrary, in a minimal culture medium (M9), greater expression times were necessary as the entire bacterial metabolism is hindered due to the poorness of the medium. In this medium, there was no problem with the appearance of yellow pigment and even, after long times, the contamination of the medium by other proteins did not increase to a great degree.

5.4 N-Igf-bp4 and Basic Fgf Production

The production of two other small proteins (N-Igf-bp4 and basis Fgf) was, for different reasons, very low, although appreciable. In the case of N-Igf-bp4 (N-Bp4) the protein underwent some kind of proteolysis when it was secreted into the medium and the form found was of lower molecular weight than expected. Although it appeared in considerable quantities, it is considered that it is not correctly expressed as almost none of the complete protein could be expressed. The complete protein was detected by the immunoblot technique (FIG. 10A). The case of basic Fgf (Fgf-b) is different as it is a protein considered from the offset as difficult to express correctly with a secretion plasmid, given that all its Cys's are free, and the red/ox character of the extracellular medium favour the formation of intramolecular (or between different molecules) disulphur bridges, and, in general, irreversible oxidations of the —SH groups which cause destabilization of the protein. Despite this, the protein expression, determined by immunoblot was appreciable (FIG. 10B). A subsequent construction starting from the pompA-basic Fgf plasmid, in which the ompA secretion sequence was eliminated, demonstrated the abovementioned as production in that case was of 30 mg/l.

The two large proteins (Homothorax and Fgf receptor) which an attempt was made to express, were not produced in appreciable quantities. In both cases, they seemed difficult to express in a soluble form. An attempt was made to express the Homothorax protein in a pET-type plasmid but, despite obtaining it, and in an apparently soluble form, there was minimum stability and it degraded until it disappeared a few hours after obtaining it (even immediately freezing it). An attempt was made to express the Fgf receptor in the pRAT plasmid without the ompA secretion sequence. A minimum amount of protein was obtained and in the form of inclusion bodies. We also tried to clone this gene in pIN-type plasmids. Not even one clone was achieved in these plasmids. All this data seems to indicate that this protein is extremely toxic for E. coli. The fact that we cannot even obtain transformer bacteria colonies for this plasmid in the pIN-type plasmids, and given that expression is not completely expressed in these type of plasmids at any time, indicate that it is so toxic that it does not even permit the growth of colonies. This detail coincides with that observed in the other clones attempted: when the culture is induced, the growth almost completely stops.

### Deposit of Biological Material

The deposits listed hereunder, corresponding to the access numbers indicated were made in the Spanish Type Culture Collection (CECT), Burjasot (Valencia):

| Culture | Access no. | Date |
|---|---|---|
| Escherichia coli | CECT 5700 | 22^{nd} March 2002 |
| BL21ss | | |
| Escherichia coli | CECT 5701 | 22^{nd} March 2002 |
| BL21(DE3)ss | | |
| Escherichia coli | CECT 5702 | 19^{th} April 2002 |
| PIN-ompA- | | |
| AM55(Bl21ss) | | |
| Escherichia coli | CECT 5703 | 19^{th} April 2002 |
| pHRO- | | |
| AM55(Bl21DE3ss) | | |

1. A plasmid which comprises:
(i) a DNA sequence that encodes for a protein that is toxic for a bacteria when said protein accumulates in the cytoplasm of said bacteria
(ii) a DNA sequence that contains a secretion sequence of a protein.

2. Plasmid, as claimed in claim 1, which further comprises a gene sequence that gives resistance to antibiotics.

3. Plasmid, as claimed in claim 2, which further comprises a repressor/activator system for the synthesis of said protein that is toxic for a bacteria when it accumulates in the cytoplasm of said bacteria.

4. Plasmid, as claimed in claim 3, in which said repressor/activator system for the synthesis of said protein that is toxic for a bacteria when it accumulates in the cytoplasm of said bacteria comprises a Lac promoter.

5. Plasmid, as claimed in claim 3, in which said repressor/activator system for the synthesis of said toxic protein comprises a promoter modulated by arabinose or tryptophan.

6. Plasmid, as claimed in claim 3, in which said repressor/activator system for the synthesis of said toxic protein comprises a promoter modulated by temperature.

7. Plasmid as claimed in claim 4, which comprises a T7 Lac RNA polymerase system that strictly regulates the synthesis of said protein that is toxic for a bacteria when it accumulates in the cytoplasm of said bacteria.

8. Plasmid, as claimed in claim 1, in which said DNA sequence (ii) is a DNA sequence that contains the ompA protein secretion sequence.

9. Plasmid, as claimed in claim 1, which further comprises a DNA sequence that contains a recognition site for a protease.

10. Plasmid, as claimed in claim 9, in which said protease is protease 3C.

11. Plasmid, as claimed in claim 1, which comprises a NaeI site immediately after the site where the peptidase, which permits secreting the protein into the culture medium, cuts.

12. Plasmid, as claimed in claim 1, selected from the group formed by pRAT-ompA-1, pRAT-ompA-2, and pRAT-ompA-3.

13. A bacteria that comprises a plasmid as claimed in any of claims 1 to 12.

14. A process to obtain a secretary bacterial strain in a minimal culture medium, which consists of:
a) transforming bacteria adapted to grow in a minimal culture medium with a plasmid as claimed in any of claims 1 to 12;
b) culturing said transformed bacteria in a minimal culture medium plus an antibiotic, under conditions that permit the expression of said protein that is toxic for a bacteria when it accumulates in the cytoplasm of said bacteria, and
c) selecting the surviving bacteria

15. Process, as claimed in claim 14, which further consists of eliminating the plasmid that encodes for said protein that is toxic for the selected surviving bacteria.

16. Process, as claimed in claim 14, in which said bacteria are a strain of E. coli adapted to grow in a minimal culture medium.

17. Process, as claimed in claim 15, in which the elimination of said plasmid is carried out by treatment with an antibiotic.

18. A bacterial strain which has the characteristics of culture deposited in the CECT with the access numbers CECT 5700, CECT 5701, CECT 5702 or CECT 5703 or a variant thereof which is capable of growing in a minimal culture medium and secreting a protein encoded for a DNA integrated in a plasmid into said medium.

19. Bacterial strain according to claim 18, derived from E. coli.

20. A bacterial strain derived from E. coli selected from:

| | |
|---|---|
| Escherichia coli BL21ss | CECT 5700 |
| Escherichia coli BL21(DE3)ss | CECT 5701 |
| Escherichia coli PIN-ompA-AM55(Bl21ss) | CECT 5702 |
| Escherichia coli pHRO-AM55(Bl21DE3ss) | CECT 5703 |

21. A process to obtain a product of interest that consists of:
a) transforming a bacterial strain in a minimal culture medium obtained by means of the process of any of claims 14 to 17, or a bacterial strain according to any of claims 18 to 20, with a plasmid that contains as DNA sequence that encodes for a product of interest.
b) Culturing said secretary bacterial strain in a minimal culture medium, transformed under conditions that permit the expression of said product of interest and its expression into the culture medium.

22. Process according to claim 21, which further comprises recovering the product of interest from the culture medium.

23. The supernatant of the bacterial culture of the process of claim 21.

24. Use of a protein that is toxic for a bacteria when it accumulates in the cytoplasm of said bacteria in the selection of the secretary bacteria.
